(19) United States
(12) Patent Application Publication
(10) Pub. No.: US 2016/0000838 A1
(43) Pub. Date: Jan. 7, 2016

(54) Use of Faecalibacterium Prausnitzii Htf-f (DSM 26943) to Suppress Inflammation

(71) Applicants: RIJKSUNIVERSITEIT GRONINGEN, Groningen (NL); ACADEMISCH ZIEKENHUIS GRONINGEN, Groningen (NL); WAGENINGEN UNIVERSITEIT, Wageningen (NL); UNIVERSITY COURT OF THE UNIVERSITY OF ABERDEEN, Aberdeen (GB)

(72) Inventors: Hermanus Josef Martinus HARMSEN, Groningen (NL); Muhammad Tanweer KHAN, Groningen (NL); Jeremy WELLS, Wageningen (NL); Oriana ROSSI, Wageningen (NL); Harry James FLINT, Aberdeen (GB); Sylvia Helen DUNCAN, Aberdeen (GB)

(73) Assignees: RIJKSUNIVERSITEIT GRONINGEN, Groningen (NL); THE UNIVERSITY COURT OF THE UNIVERSITY OF ABERDEEN, Aberdeen (GB); WAGENINGEN UNIVERSITEIT, Wageningen (NL); ACADEMISCH ZIEKENHUIS GRONINGEN, Groningen (NL)

(21) Appl. No.: 14/772,519

(22) PCT Filed: Mar. 5, 2014
(86) PCT No.: PCT/NL2014/050131

§ 371 Date: Sep. 3, 2015

Related U.S. Application Data
(60) Provisional application No. 61/778,458, filed on Mar. 13, 2013.

(30) Foreign Application Priority Data
Mar. 5, 2013 (EP) 13157830.4

(51) Int. Cl. A61K 35/741; A23L 1/03; A61K 45/06

(52) U.S. Cl. CPC A61K 35/741; A61K 45/06; A23L 1/0345; A23V 2002/00

(57) ABSTRACT

The present invention relates to medicine, particularly immunology and gastroenterology. Specifically, it relates to probiotic bacteria and extracts thereof for therapeutic use for the treatment of inflammatory disorders such as inflammatory bowel disease. Provided is a composition comprising as active ingredient Faecalibacterium prausnitzii strain HTF-F (DSM 26943) or an extract thereof comprising extracellular polymeric matrix (EPM), and an acceptable carrier, diluent or excipient. Also provided is an anti-inflammatory composition comprising EPM extracted from F. prausnitzii strain HTF-F, and a method for preparing the same.

The present invention relates to medicine, particularly immunology and gastroenterology. Specifically, it relates to probiotic bacteria and extracts thereof for therapeutic use for the treatment of inflammatory disorders such as inflammatory bowel disease and diseases related to microbiota disbalance. Also provided are anti-inflammatory (probiotic) compositions, e.g. functional foods, neutraceuticals or pharmaceutical compositions, and methods for producing them, as well as approaches for promoting probiotic survival in the gut.

Inflammatory bowel disease (IBD) is a chronic inflammatory disorder of the gastrointestinal tract characterized by diarrhoea, bloody stools, abdominal pain and weight loss. IBD is a chronic, relapsing inflammation of unknown origin. IBD comprises two main disease conditions, ulcerative colitis (UC) and Crohn's disease (CD). UC affects mostly the mucosal layer of the large intestine or colon. In contrast, CD is defined as a transmural granulomatous inflammation that can involve segments of the small and large intestine. UC and CD are driven by an aberrant inflammatory T cell response to intestinal microbiota in a genetically susceptible host. Profound changes in the diversity and composition of the microbiota are associated with UC and CD (Seksik, Rigottier-Gois et al. 2003; Macfarlane, Blackett et al. 2009). A decrease in the frequency of the phyla Bacteroidetes and Firmicutes and an increase of Proteobacteria and Actinobacteria has been observed in the faecal microbiota of IBD patients (Frank, St Amand et al. 2007). In particular, Faecalibacterium prausnitzii, a member of the Firmicutes phylum and one of the most abundant species in the healthy human colon (Flint, Scott et al. 2012), is underrepresented in the microbiota of IBD patients (Sokol, Seksik et al. 2009). Mucosal-associated counts of F. prauznitzii from ileal biopsies are also lower in CD patients with active disease than in patients in remission (Sokol, Pigneur et al. 2008).

F. prausnitzii was reported to be an anti-inflammatory bacterium on account of its capacity to induce high amounts of IL-10 in human peripheral blood mononuclear cells (PBMCs, (Sokol, Pigneur et al. 2008)), and dendritic cells. Furthermore, treatment of Caco-2 cells with F. prausnitzii culture supernatant was reported to reduce IL-1β-induced NF-kB activation and secretion of IL-8. This was attributed to an as yet unidentified factor secreted in the medium (Sokol, Pigneur et al. 2008). Additionally, administration of F. prausnitzii strain A2-165 and its culture supernatant have been shown to protect against 2,4,6-trinitrobenzenesulfonic acid (TNBS)-induced colitis in mice (Sokol, Pigneur et al. 2008). This model is thought to resemble CD because the resulting mucosal inflammation is mediated by a T helper 1 (Th1) response with excessive production of IFN-γ, TNF-α and IL-12.

The present inventors recognized the need for further F. prausnitzii strains for use in the prophylaxis and/or treatment of inflammatory disorders. They specifically aimed to identify a strain which is more effective than strain A2-165 (DSMZ 17677) used in the prior art. A further goal was to provide a bacterial extract having anti-inflammatory properties. To that end, the capacity of different strains of F. prausnitzii and extracts thereof to suppress inflammation in the mouse dextran sodium sulphate (DSS) colitis model was evaluated.

It was found, surprisingly, that living cells of F. prausnitzii strain HTF-F as well as the extracellular polymeric matrix (EPM) isolated therefrom have strong immune-regulating properties. This strain was deposited by the present applicant with the Deutsche Sammlung von Mikroorganismen and Zellkulturen (DSMZ) on Mar. 1, 2013 under the accession number DSM 26943. Strain HTF-F was previously reported by Lopez-Siles et al. (2012) as one of several new isolates obtained from healthy human donors. Based on 16S rRNA sequence analysis it was found to belong to phylogroup II. However, no therapeutic use of strain HTF-F has ever been reported or suggested in the art, let alone its strong anti-inflammatory effects in IBD.

Importantly, the anti-inflammatory effect of HTF-F was found to be more effective than that of strain A2-165, partly because of the immune-regulating properties of the EPM of HTF-F. The immunomodulatory effects of the EPM are mediated at least partially through the TLR2-dependent modulation of IL-12 and IL-10 cytokine production in antigen presenting cells. It was also observed that strain HTF-F undergoes intercellular aggregation and forms a dense biofilm in liquid culture, while strain A2-165 does not. Without wishing to be bound by theory, the biofilm might provide some advantage to bacterial colonization in vivo. In particular, EPM may protect this bacterium against oxidative stress and therefore it can reside close to the gut epithelium which is the main source of oxygen via inward diffusion, better than strains that produce less EPM. This would help to deliver butyrate and other anti-inflammatory products close to the colonic wall. All these features make F. prausnitzii strain HTF-F a robust probiotic to treat colitis and other inflammatory gut diseases. Hence, both F. prausnitzii HTF-F and its EPM are advantageously used as therapeutics in the management of IBD and related inflammatory diseases.

Accordingly, in one embodiment the invention provides a composition comprising as active ingredient Faecalibacterium prausnitzii strain HTF-F or an extract thereof comprising extracellular polymeric matrix, and an acceptable carrier, diluent or excipient. The composition is for instance a pharmaceutical composition, a food composition or a neutraceutical composition. Also provided is F. prausnitzii strain HTF-F DSM 26943 (hereinafter also referred to as “HTF-F”) and/or extract thereof comprising extracellular polymeric matrix for use as a medicament. For example, HTF-F or an HTF-F extract comprising extracellular polymeric matrix is advantageously used in a method for treating or preventing an inflammatory disorder of the gastrointestinal tract of a mammalian subject. This is illustrated herein below by the attenuation of clinical parameters in a murine colitis model.

Also provided is a method for treating or preventing symptoms associated with an inflammatory disorder of the gastrointestinal tract of a mammalian subject, comprising the administration of an effective amount of Faecalibacterium prausnitzii strain HTF-F or extract thereof comprising EPM.

According to the invention, the Faecalibacterium prausnitzii strain may be in the form of viable cells. Alternatively, they are in the form of non-viable cells. Mixtures of viable and non-viable cells are also envisaged. The general use of probiotic bacteria is in the form of viable cells. However, it can also be extended to non-viable cells such as killed cultures or compositions containing beneficial factors expressed by the probiotic bacteria. This could include thermally killed micro-organisms or micro-organisms killed by exposure to altered pH or subjection to pressure or gamma irradiation. Cells may be incorporated into pharmaceuticals. With non-viable cells, product preparation is simpler and storage requirements are much less limited than viable cells, in particular in view of its oxygen-sensitivity.

In a specific aspect, living bacteria or EPM is administered directly in the gastrointestinal tract, for example by intrarectal delivery. Other options for non-bacteria delivery include gut-independent pathways, including systemic delivery such as by interperitoneal injection. In a preferred embodiment of the invention, one or more purified anti-inflammatory component(s) of the bacteria which does not elicit inflammatory responses in host cells is administered, e.g. orally, subcutaneously or topically.

In view of its strong anti-inflammatory effects, the invention finds its use in various pathologies involving unwanted inflammatory responses. Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

In one embodiment, the inflammatory disease is an inflammatory gastrointestinal disorder, preferably selected from the group consisting of inflammatory bowel disease, Crohn's disease, irritable bowel syndrome, ulcerative colitis, coeliac disease, infectious colitis (e.g, caused by C. difficile), ulcerative colitis, other gut-related diseases, and any combination thereof. Alternatively, the inflammatory disease is an inflammatory autoimmune disorder wherein a dys-balanced microbiota and a low grade mucosal inflammation is related to the aetiology or onset of the disease, such as diabetes (type 1 and 2), asthma and atopic diseases.

In a further aspect, strain HTF-F and compositions comprising them are used as preventive or curative agent in a person suffering from or at increased risk of developing a condition or disorder related to a gut microbiota disbalance, The gut microbiota composition has been associated with several hallmarks of metabolic syndrome (e.g., obesity, type 2 diabetes, cardiovascular diseases, and non-alcoholic steatohepatitis). Growing evidence suggests that gut microbes contribute to the onset of the low-grade inflammation characterising these metabolic disorders via mechanisms associated with gut barrier dysfunctions. Recently, enteroendocrine cells and the endocannabinoid system have been shown to control gut permeability and metabolic endotoxaemia. Moreover, targeted nutritional interventions using non-digestible carbohydrates with prebiotic properties have shown promising results in pre-clinical studies in this context, although human intervention studies warrant further investigations. Thus, a composition of the invention is advantageously used to establish or maintain a balanced gut microbiota. Furthermore, HTF-F can serve to change the gut microbiota as a therapeutic target in the context of obesity and type 2 diabetes.

The connection between gut microbiota and energy homeostasis and inflammation and its role in the pathogenesis of obesity-related disorders are increasingly recognized. Animals models of obesity connect an altered microbiota composition to the development of obesity, insulin resistance, and diabetes in the host through several mechanisms: increased energy harvest from the diet, altered fatty acid metabolism and composition in adipose tissue and liver, modulation of gut peptide YY and glucagon-like peptide (GLP)-1 secretion, activation of the lipopolysaccharide toll-like receptor-4 axis, and modulation of intestinal barrier integrity by GLP-2.

In a preferred embodiment, strain HTF-F or extract thereof comprising anti-inflammatory agents are used to protect for or treat inflicted harm to the mucosal layer of the large intestine or colon.

Preferably, the subject to be treated is a human subject. For example, it is a human subject known or suspected to suffer from ulcerative colitis. However, the invention is also applicable in veterinary medicine. For example, it also provides Feacalibacterium prausnitzii strain HTF-F or extract thereof for therapeutic use in a domestic animal or an agricultural animal.

F. prausnitzii strain HTF-F or extract thereof preferably causes an increase in the anti-inflammatory cytokine production and/or decreases production of one or more pro-inflammatory cytokine in the subject. For instance, the anti-inflammatory cytokine is IL-10, or TGF-beta. In one embodiment, pro-inflammatory cytokine is dependent on TLR2 signalling, preferably said cytokine is IL-12p70.

We also describe F. prausnitzii strain HTF-F or extract thereof for use in the preparation of an anti-inflammatory biotherapeutic agent for the prophylaxis and/or treatment of undesirable inflammatory activity or for use in the preparation of anti-inflammatory biotherapeutic agents for the prophylaxis and/or treatment of undesirable inflammatory activity. The strain may act by antagonising and excluding proinflammatory micro-organisms from the gastrointestinal tract.

The bacterium or extract is formulated to be administered to a subject in a therapeutically effective amount, depending on e.g. type of subject, disease severity and route of administration. Typically, the therapeutically effective amount of the bacterium is about 10exp6 to 10exp11 CFU/day, preferably 10exp7 to 10exp10 CFU/day.

The bacteria can be administered via any suitable route of administration. For example, the specific Faecalibacterium prausnitzii strain of the invention may be administered to animals (including humans) in an orally ingestible form. In case of a food composition or neutraceutical, the bacteria can simply be incorporated in a conventional food item or food supplement. Exemplary pharmaceutical formulations include capsules, microcapsules, tablets, granules, powder, troches, pills, suppositories, suspensions and syrups. In another embodiment, the composition is in a form for rectal administration to an animal (including humans), for instance as rectal suppository or enema. Suitable formulations may be prepared by methods commonly employed using conventional organic and inorganic additives. The amount of active ingredient in the medical composition may be at a level that will exercise the desired therapeutic effect.

The composition may contain any useful further ingredients such as ingredients that are known to support the growth or maintenance of beneficial bacteria so as to modify the gastrointestinal microbial community in a beneficial manner. Such ingredients are called “prebiotics.” Typical examples of known prebiotics are oligosaccharides, such as fructooligosaccharides and inulin. Synbiotics refer to nutritional supplements combining probiotics and prebiotics in a form of synergism.

F. prausnitzii bacteria needed for the afore-mentioned anti-inflammatory effects are extremely sensitive to oxygen and cannot survive exposure to ambient air for more than a few minutes. As a consequence, probiotic compositions containing viable F. prausnitzii have not been described thus far despite their promising therapeutic application.

A specific aspect of the invention relates to a synbiotic composition comprising living cells of E prausnitzii strain HTF-F mixed with riboflavin, riboflavin phosphate or a physiologically acceptable salt thereof, and cysteine. This mixture was found to give a surprisingly good protection of these bacteria against exposure to ambient air during manufacturing, storage and/or consumption. For example, it can withstand ambient air exposure for at least 24 h, and is stable in simulated gastrointestinal fluid for at least 2 h. Also, the synbiotic formulation according to the invention can be stable for at least 2 h when mixed with a food product, for instance milk or yoghurt drinks. Preferably, riboflavin is used.

The riboflavin, riboflavin phosphate or physiologically acceptable salt thereof, is present in an amount of at least 0.05%, preferably at least 1%, more preferably at least 2% based on the total dry weight of the composition. For example, it may contain 0.05 to 10%, preferably 1-10%, like 2-10%, or 0.05-0.25% based on the total dry weight of the composition. Cysteine is preferably present in an amount of at least 0.05% based on the total dry weight of the composition. A cysteine content up to 2% is suitably used. For example, the composition may contain 0.1-1.5%, 0.5-1% or 0.05-0.2% cystein based on the total dry weight of the composition.

The composition may further comprise one or more prebiotics, preferably oligosaccharides, more preferably fructooligosaccharides, pectins and/or inulin orinulin-type fructo-oligosaccharides, preferably in an amount of 2-10% based on the total dry weight of the composition. Other suitable ingredients include bulking agents, preferably in an amount of 40-65% based on the total dry weight of the composition.

Of course, the composition may contain further useful ingredients, including further prebiotics and/or probiotics. Useful probiotic bacteria are preferably selected from the group consisting of lactic acid bacteria, bifidobacteria or mixtures thereof. Probiotic bacteria may be any lactic acid bacteria or bifidobacteria with established probiotic characteristics. For example, they may be also capable of promoting the development of a bifidogenic intestinal microbiota. In some cases they might also help to protect F. prausnitzii against oxygen. Suitable additional probiotics may be selected from the group consisting of Bifidobacterium, Lactobacillus, Streptococcus and Saccharomyces or mixtures thereof, in particular selected from the group consisting of Bifidobacterium longum, Bifidobacterium lactis, Lactobacillus acidophilus, Lactobacillus rhamnosus, Lactobacillus paracasei, Lactobacillus johnsonii, Lactobacillus plantarum, Lactobacillus salivarius, Enterococcus faecium, Saccharomyces boulardii and Lactobacillus reuteri or mixtures thereof, preferably selected from the group consisting of Lactobacillus johnsonii (NCC533; CNCM 1-1225), Bifidobacterium longum (NCC490; CNCM 1-2170), Bifidobacterium longum (NCC2705; CNCM 1-2618), Bifidobacterium lactis (2818; CNCM 1-3446), Lactobacillus paracasei (NCC2461; CNCM 1-2116), Lactobacillus rhamnosus GG (ATCC53103), Lactobacillus rhamnosus (NCC4007; CGMCC 1.3724), Enterococcus faecium SF 68 (NCIMB10415), and mixtures thereof. In one embodiment, the further probiotic comprises food-grade bacteria which may be recombinant non-pathogenic food-grade bacteria serving as delivery vehicles of an anti-inflammatory molecule. See for example WO2011/086172 and references cited therein. Useful growth substrates include cellobiose and lactulose. The formulation may contain fillers and extenders, such as maltodextrin or pullulan. In one aspect, the composition comprises a consortium of anaerobic bacteria including strain HTF-F. These anaerobic bacteria are providing essential nutrients like sugars, amino acids, acetate and vitamins like riboflavin to HTF-F, to facilitate and enhance its growth. For instance, bifidobacteria and members of the Clostridium groups IV and XIVa are suitably used in combination with strain HTF-F.

In another specific aspect, the invention provides a composition comprising extracellular polymeric matrix extracted from Faecalibacterium prausnitzii strain HTF-F, and the use thereof in therapy, in particular as an anti-inflammatory agent. Also provided is a method for producing such anti-inflammatory extract. In one embodiment, the method, comprises the steps of: a) harvesting living cells of F. prausnitzii strain HTF-F and resuspending the cells in a suitable buffer, preferably PBS; b) vortexing the resuspended cells, preferably for at least 3 minutes, allowing the cell-bound EPM to dissolve in the buffer; c) pelleting the cells by centrifugation and harvesting the supernatant; d) adding about 4 volumes of ice-cold ethanol to the supernatant to precipitate the extracellular polymeric matrix (EPM); e) washing the precipitated EPM with ethanol; and f) followed by lyophilisation (optional). The (lyophilized) precipitate may be reconstituted at a desirable concentration in any suitable carrier prior to use. For example, it is dissolved in saline such as PBS.

An extract of the invention is capable of modulating the production of at least one immunomodulatory cytokine. In one embodiment, it induces in an in vitro system an increase in the anti-inflammatory cytokine production and/or a decrease in the production of one or more proinflammatory cytokine. For example, the extract is tested in vitro using human monocyte derived dendritic cells (HMDCs) or murine bone-marrow-derived dendritic cells (BMDCs), optionally stimulated with L. plantarum. Alternative assays include those employing peripheral blood mononuclear cells from human blood or colonic cultures, for example cytokine production can be measured in cultured colonic fragments obtained from DSS-treated mice.

In one embodiment, the extract increases the production of anti-inflammatory cytokine IL-10. In another embodiment, the extract decreases the production of one or more proinflammatory cytokine, like IL-12, IL-17 and/or IFNgamma. The reduction of pro-inflammatory cytokine for example IL-12p70, may be dependent on TLR2 signalling. In a preferred embodiment, the extract induces anti-inflammatory cytokine production and reduces pro-inflammatory cytokines.

Also provided is a method for treating or preventing symptoms associated with an inflammatory disorder of the gastrointestinal tract of a mammalian subject, comprising administering F. prausnitzii strain HTF-F or an anti-inflammatory extract thereof comprising EPM. The gastrointestinal disorder is selected from the group consisting of inflammatory bowel disease, Crohn's disease, irritable bowel syndrome, coeliac disease, infectious colitis, ulcerative colitis, and any combination thereof. Preferably, the subject to be treated is a human. F. prausnitzii strain HTF-F is administered to a subject in a therapeutically effective amount, typically wherein the therapeutically effective amount of the bacterium is about 10exp6 to 10exp11 CFU/day. In a specific embodiment, the bacteria are administered as being part of a synbiotic composition described herein above, comprising living cells of Faecalibacterium prausnitzii strain HTF-F formulated with riboflavin, riboflavin phosphate or a physiologically acceptable salt thereof, and cysteine.

### LEGEND TO THE FIGURES

FIG. 1: a) Growth and biofilm formation of F. prausnitzii strains HTF-F and A2-165 in YCFAG medium under anaerobic conditions. i and ii) F. prausnitzii A2-165 before and after shaking respectively; iii and iv) F. prausnitzii HTF-F before and after shaking respectively. b) Gram staining of F. prausnitzii A2-165 (left panel) and HTF-F (right panel).

FIG. 2: Detection of F. prausnitzii HTF-F EPM by transmission electron microscopy. F. prausnitzii HTF-F (a) possess a diffuse and irregular surface layer (arrow) which is thinner but similar to the capsule polysaccharide (CPS) of S. suis wild type strain (arrow, S. suis wt, left panel b) and absent in S. suis CPS deletion mutant (S. suis without cps, right panel b).

FIG. 3: TLR signalling properties of F. prausnitzii HTF-F EPM. NF-kB activation was measured using a luminescence reporter in HEK293 cell lines expressing TLR2/1, TLR2/6, TLR5 and TLR4, relative values were calculated deducting the medium control values to the measured values. Error bars represent SEM, n=6.

FIG. 4: Cytokine secretion and surface marker expression in human immature dendritic cells (hDCs) after 48 h of incubation with F. prausnitzii A2-165 (3 donors), F. prausnitzii HTF-F (3 donors), L. plantarum (5 donors), L. plantarum+EPM (5 donors), EPM (3 donors) or left unstimulated (5 donors). a) IL-10 and IL-12p70 were measured in the supernatant of hDCs. Error bars represent SEM, * indicates p<0.05 compared with L. plantarum treated samples. b) Percentage of CD83⁺ (left panel) and CD86⁺ (right panel) hDCs. Error bars represent SEM, ** indicates p<0.01, n.s. indicates non significant compared with the control.

FIG. 5: Relative gene expression levels in hDCs determined by quantitative RT-PCR. RNA was extracted from hDCs after 6 and 20 h of incubation with L. plantarum (in black), L. plantarum+EPM (in dark grey), EPM (in clear grey) or from unstimulated cells (in white) and the expression levels of IL-12p70 gene was calculated relative to the expression levels of the housekeeping gene GAPDH. Error bars represent SEM, n=3, *** indicates p<0.001 compared with L. plantarum treated samples.

FIG. 6: Disease activity index (DAB, colon histological damage score and clinical evaluation of DSS treated mice. Mice were left untreated (in white) or treated with DSS during 8 days and administered intrarectally with PBS (in black), EPM (in blue) or F. prausnitzii strains HTF-F (in red) or strain A2-165 (in green). DAI, histological score and colon length (a, b and c respectively) were evaluated at the end of the experiment. Mice body weight (d) was measured throughout the experiment, body weight values are expressed as percentage of the initial value measured at day 0 before DSS administration. Error bars represent SEM, n=10, * indicates p<0.05, ** p<0.01, *** p<0.001 compared with the control colitis mice that received DSS+PBS.

FIG. 7: Histological cross-sectional views of colon descendens of untreated or DSS-treated mice: a) colitis control, PBS-DSS-treated mice (damage grade 3-3.5) b) HTF-F-DSS-treated mice (damage grade 1-2.4); c) A2-165-DSS-treated mice, (damage grade 2-3.7); d) EPM-DSS-treated mice (damage grade 2.8-3.8); e) untreated mice (damage grade 0).

FIG. 8: Percentage of Foxp3⁺ CD4⁺ T cells isolated from mesenteric lymph nodes (MLNs, left panel) and spleens (right panel) of mice untreated (in white) or treated with DSS during 8 days and administered intrarectally with PBS (in black), EPM (in blue) or F. prausnitzii strains HTF-F or strain A2-165 (HTF-F in red and A2-165 in green, respectively).

FIG. 9: Cytokine secretion in mouse BMDCs. a) IL-10 and IL-12p70 were measured in BMDC supernatants after incubation with L. plantarum, L. plantarum+EPM, EPM and unstimulated DCs. b) IL-10 and IL-12p70 were measured after incubation of BMDCs with the same samples as in panel (a) except that anti-TLR2 blocking antibody (anti-TLR2 Ab, dark grey bars) or an isotype control (isotype Ab, clear grey bars) were included during the incubation period. Error bars represent SEM (n=3), *** indicates p<0.001, * indicates p<0.01, n.s. non significant compared to L. plantarum treated samples.

FIG. 10: Cytokine secretion in colon cultures from DSS treated mice. Mice were left untreated or treated with DSS during 8 days and administered intrarectally with PBS, EPM or F. prausnitzii strain HTF-F or A2-165. Cytokines were measured in the supernatants of 48 h cultures of colonic fragments isolated from mice. Error bars represent SEM, n=5, * indicates p<0.05, ** p<0.01.

FIG. 11: IL-12p70 secretion in human immature dendritic cells (hDCs) from 2 different donors after 48 h of incubation with L. plantarum, L. plantarum+EPM, L. plantarum+ extracts from uncultured bacterial medium (control EPM), EPM, extracts from uncultured bacterial medium (control EPM) or left unstimulated (2 donors). The EPM from F. prausnitzii HTF-F attenuated IL-12 secretion. This effect was not observed using control extracts from uncultured medium. The EPM extracts, control extracts from uncultured medium, and medium control did not induce IL-12 secretion or other cytokines (not shown). These results show that the effect of the EPM is not due a component of the uncultured medium.

### EXPERIMENTAL SECTION

### Materials and Methods

Animals

BALB/c mice were reared in conventional conditions. Two-month-old females were used for these studies and their body weights were measured before and after each experiment. Animal experiments were approved by the Ethical Committee of the Institute of Microbiology, Academy of Sciences of the Czech Republic.

Bacterial Strains and Culturing Conditions

F. prausnitzii strain HTF-F and A2-165 have been described elsewhere (Barcenilla, Pryde et al. 2000; Duncan 2002; Lopez-Siles, Khan et al. 2012) and were maintained at 37° C. on yeast extract, casitone, fatty acid and glucose medium (YCFAG, described in (Lopez-Siles, Khan et al. 2012)) under anaerobic conditions. Strain HTF-F was deposited with the Deutsche Sammlung von Mikrooganismen and Zellkulturen (DSMZ) on Mar. 1, 2013 under the accession number DSM 26943

YCFA medium consists of (per 100 ml) Casitone (1.0 g), yeast extract (0.25 g), NaHCO₃ (0.4 g), cysteine (0.1 g), K₂HPO₄ (0.045 g), KH₂PO₄ (0.045 g), NaCl (0.09 g), (NH₄)₂SO₄ (0.09 g), MgSO₄.7H₂O (0.009 g), CaCl₂ (0.009 g), resazurin (0.1 mg), hemin (1 mg), biotin (1 microgram), cobalamin (1 microgram), p-aminobenzoic acid (3 microgram), folic acid (5 microgram), and pyridoxamine (15 microgram). In addition, the following short-chain fatty acids (SCFA) are included (final concentrations): acetate (33 mM); propionate (9 mM); isobutyrate, isovalerate, and valerate (1 mM each). Cysteine is added to the medium following boiling and dispensed into Hungate tubes while the tubes are flushed with CO2. After autoclaving, filter-sterilized solutions of thiamine and riboflavin are added to give final concentrations of 0.05 microgram/ml of each.

For EPM production, F. prausnitzii strains were cultured in YCAG broth, which have the same composition as YCFAG medium but, except acetate, all short chain fatty acids were omitted. L. plantarum WCFS1 was cultured overnight until stationary phase in deMan, Rogosa Sharpe broth (MRS, Merck, Darmstadt, Germany) at 37° C. Bacteria were harvested by centrifugation at 4° C., 3300 g for 15 min, washed in phosphate buffer saline (PBS), resuspended in PBS containing 20% glycerol and stored at −80° C. prior to use. For the BMDCs assays, L. plantarum WCFS1 grown in MRS at 37° C. overnight were inactivated with 1% formaldehyde-PBS as described previously (Schabussova, Hufnagl et al. 2012).

Bacteria were quantified by fluorescent in situ hybridization (FISH) or phase contrast microscopy. All buffers and media used for the anaerobic bacteria were deoxygenated by flushing with oxygen free nitrogen for 30 minutes.

Isolation and Staining of the F. prausnitzii Extracellular Polymeric Matrix

The cell bound EPM was extracted as previously described (Ricciardi, Parente et al. 1998). 250 ml of 24 h old cultures of F. prausnitzii were recovered by centrifugation at 3300 g for 15 min, washed in PBS and followed by centrifugation step. The pre-washed cell pellet was suspended in 8 ml of PBS by vortexing for 5 min allowing the cell-bound EPM to dissolve. Cells were subsequently pelleted by centrifugation at 18400 g for 10 min (4° C.). The supernatant was then carefully harvested and added to 4 volumes of ice cold absolute ethanol to precipitate the EPM. After centrifugation at 3300 g for 30 min, the EPM precipitated pellet was washed with 70% ethanol, then lyophilized and stored at −20° C. For further experiments, lyophilized EPM fractions were dissolved in PBS at the desired concentrations. The EPM was shown to be free of bacterial contamination by visual inspection after Gram staining TLR assays (described in the following section) showed that the EPM was free of contaminating MAMPs.

TLR Signalling Assays

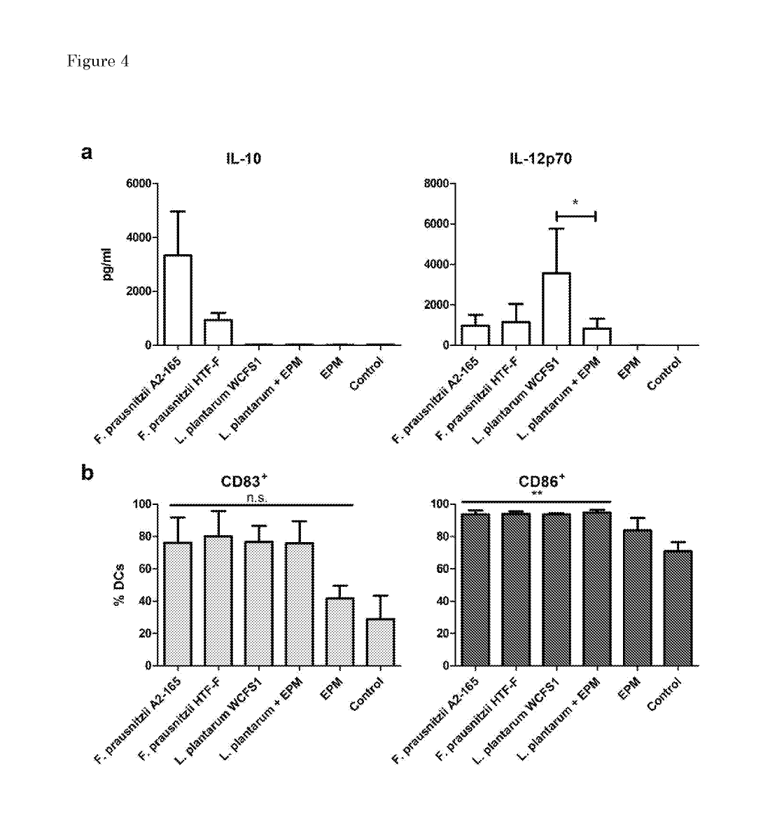

TLR assays were performed using human embryonic kidney cells (HEK293) stably expressing human TLR2/6, TLR2/1, TLR4 or TLR5 (Invivogen, Toulouse, France) and transfected with a reporter plasmid (pNiFTY, Invivogen) containing the luciferase gene under the control of the NF-kB promoter. HEK293 cells expressing the different TLRs and pNiFTY were incubated with the EPM (1.2% v/v), the TLR agonists, Pam2CSK4 (Invivogen) for TLR2/6, Pam3CSK4 (Invivogen) for TLR2/1, flagellin (Invivogen) for TLR5 and LPS for TLR4, or medium alone as a control. After 6 hours of incubation, the medium was replaced with Bright glow (Promega), and the luminescence was measured using a Spectramax M5 (Molecular Devices). As a negative control, HEK293 cells not expressing TLRs but harbouring pNiFTY were tested in the same conditions and did not show any luciferase activity. The limits of sensitivity for the TLR reporter cell lines were determined in independent experiments using a dose range for each purified agonist. The limits of detection for these reporter cell lines were determined as follows: 2 ng/ml for Pam2CSK4 (TLR2), 8 ng/ml for flagellin (TLR5) and 50 pg/ml for LPS (TLR4); MAMPs present at concentrations lower than this would not activate immune cells in our in vitro assays and, as expected, the EPM did not activate hDCs or mouse BMDCs (FIGS. 4 and 9).

Human DCs Assays

This study was approved by Wageningen University Ethical Committee and was performed according to the principles of the Declaration of Helsinki. Buffy coats from blood donors were obtained from the Sanquin Blood bank in Nijmegen (The Netherlands). A written informed consent was obtained before sample collection. The immunomodulatory properties of the EPM was investigated by measuring the expression of surface markers and the cytokines secreted in the supernatant after incubation of human monocyte-derived DCs (hDCs) with F. prausnitzii A2-165 (hDCs from 3 donors), F. prausnitzii HTF-F (hDCs from 3 donors), L. plantarum (hDCs from 5 donors), EPM (hDCs from 3 donors) or L. plantarum together with EPM (hDCs from 5 donors). Bacteria were used at a bacterium: DC ratio of 10:1, EPM at 1.2% v/v. Mononuclear cells were isolated from buffy coats of healthy donors using Ficoll Paque Plus density gradient (GE Healthcare, Diegem Belgium) according to the manufacturer's protocol. After centrifugation, mononuclear cells were collected and monocytes were isolated by positive selection of CD14⁺ cells using CD14-specific antibody coated magnetic microbeads (Miltentyi Biotec, Leiden, The Netherlands). CD14⁺ cells were cultured for 6 days in complete medium in the presence of IL-4 and granulocyte-macrophage colony-stimulating factor (GMCFS, R&D Systems, Minneapolis, Minn.) to differentiate into immature monocyte-derived DCs. At day 6, cells were seeded at 10⁶ cells/well in 24-well plates and were treated with L. plantarum (bacterium: DC, 10:1) in the presence or absence of the EPM isolated from F. prausnitzii (1,2% v/v) or left untreated. After 48 hours of co-incubation, the supernatant was collected for cytokine measurements.

During the culture period and the stimulation, DCs were cultured in Rosewell Park Memorial Institute (RPMI) 1640 culture medium (Invitrogen) supplemented with 10% FCS, 100 U/ml penicillin and 100 μg/ml streptomycin (Sigma, St. Louis, Mo.) and no bacterial growth was observed. On day 6 and 8, the activation and maturation status of the CD14⁺ cells were assessed by measuring CD83 and CD86 surface expression and the cell viability was measured using Annexin V and propidium iodide (PI). Cells were stained with fluorescence conjugated monoclonal antibodies specific for CD83, CD86, their isotype-matched controls and with annexin V and PI (BD Biosciences, Breda, The Netherlands) and analysed on a flow cytometer (FACS Canto II, BD). CD86 and CD83 were expressed at low levels on immature or untreated DCs and were highly expressed after stimulation. On days 6 and 8 the viability of the cells was between 60-80% (not shown).

RNA Isolation and Real-Time qPCR

Total RNA was isolated from hDCs using the RNAeasy Mini Kit (Qiagen, Venlo, The Netherlands) following the manufacturer instructions. cDNA synthesis was performed using 500 ng of isolated total RNA and Q-script (Quanta bioscience, Gaithersburg, Md.) according to the manufacturer instructions. cDNA was diluted in nuclease-free water to a final volume of 100 μl and stored at −20° C. until further use. Primers for IL-10 (forward 5′-GTGATGCCCCAAGCTGAGA-3′, reverse 5′-CACGGCCTTGCTCTTGTTTT-3′), IL-12p40 (forward 5′-CTCTGGCAAAACCCTGACC-3′, reverse 5′-GCTTAGAACCTCGCCTCCTT-3′), IL-1β (forward 5′-GTGGCAATGAGGATGACTTGTTC-3′, reverse 5′-TAGTGGTGGTCGGAGATTCGTA-3′), TNF-alpha (forward 5′-CTGCTGCACTTTGGAGTGAT-3′, reverse 5′-AGATGATCTGACTGCCTGGG-3′), and the reference genes GAPDH (forward 5′-CTGCACCACCAACTGCTTAG-3′, reverse 5′-GTCTTCTGGGTGGCAGTGAT-3′) and beta-actin (forward 5′-TTGCGTTACACCCTTTCTTG-3′, reverse 5′-CACCTTCACCGTTCCAGTTT-3′) were designed using PRIMER3 software (Rozen and Skaletsky, 2000). Quantitative RT-PCR (qPCR) was performed using the GoTaq qPCR mastermix (Promega), briefly, 5 μl cDNA (20× dilution), forward and reverse primers (300 nM each) were added to 7 μl qPCR mastermix and demineralised water was added to a final volume of 14 μl. The qPCR reaction (2 min 95° C., 40 cycles of 15 s at 95° C., 60 s at 60° C.) was carried out on a Rotorgene 6000 real-time cycler (Qiagen). Raw data were analysed using the comparative quantitation method of the Rotor-gene Analysis Software V5.0 and relative gene expression levels were determined as ratio of target gene vs. reference gene and were calculated according to the ΔCt method described by Pfaffl (Pfaffl 2001) using the following equation: Ratio=(E_{target}) Ct_{target} (control−sample)/(E_{reference}) Ct_{reference}(control−sample). Where E is the amplification efficiency and Ct is the number of PCR cycles needed for the signal to exceed a predetermined threshold value. Dual internal reference genes (GAPDH and β-actin) were incorporated in all qPCR experiments and results were similar following standardization to either gene. For each sample a controls that was not treated with reverse transcriptase was included and no amplification above background levels was observed. Non-template controls were included for each gene in each run and no amplification above background levels was observed. Specificity of the amplification was ensured by checking the melting temperature and profile of each melting curve. The product of each template was checked at least once by sequencing.

Mouse BMDCs Assays

Mouse BMDCs from BALB/c mice were prepared as previously described (Lutz, Kukutsch et al. 1999). Briefly, bone marrow cells isolated from femurs and tibias were seeded at 2×10⁵ cells/ml in bacteriological Petri dishes in RPMI 1640 medium containing 10% fetal bovine serum (FBS), 150 μg/ml gentamycin and 20 ng/ml mouse rGM-CSF (Sigma-Aldrich, USA). Fresh medium was added at day 3 and 6 and BMDC were used on day 8 of culture. Where indicated BMDCs (10⁶ cells/ml) were incubated with anti-TLR2 antibody (InvivoGen, USA) or control isotype antibody IgG2a (eBioscience, USA) at concentration 10 μg/ml for 1 hour at 37° C. prior to stimulation with L. plantarum, EPM or L. plantarum together with EPM for 20 h. L. plantarum was used at a bacterium: DC ratio of 10:1, EPM at 1.2% v/v. Culture supernatants of stimulated BMDC were stored at −20° C. until use. For cell surface marker analysis, BMDCs were collected after cultivation and pre-incubated with anti-mouse CD16/CD32 (eBioscience, USA) for 5 min on ice prior to staining for 30 min at 4° C. with anti-mouse FITC-conjugated CD11c, APC-conjugated MHCII and PE-conjugated CD40, CD80 or CD86 monoclonal antibody (eBioscience, USA). The sample data were acquired on a FACSCalibur flow cytometer (Becton-Dickinson, USA) and analyzed with FlowJo software 7.6.2 (TreeStar, USA).

Cytokine Analysis

Cytokine concentrations in the hDC culture supernatants were determined using Cytokine bead arrays (BD) and a flow cytometer (FACS Canto II, BD). In hDCs studies, the limits of detection were as follows: IL-1beta7.2 pg/ml, IL-10 3.3 pg/ml, TNF 3.7 pg/ml and IL-12p70 1.9 pg/ml. Mouse IL-10 was assayed in culture supernatants by enzyme-linked immunosorbent assay (ELISA) using Ready-Set-Go! kit (eBioscience, USA) according to manufacturer's instructions. Levels of IL-12p70 were measured with matched antibody pairs (BD Pharmingen, USA).

Intrarectal Administration of Bacteria or the EPM and Induction of Acute Ulcerative Colitis

The experimental groups of 10 mice and their respective treatments are shown in Table 1. Mice from groups 2, 3, 4 and 5 received 2.5% DSS (molecular weight 40 kDa; ICN Biomedicals, Ohio, USA) in the drinking water ad libitum for one week. Mice from the untreated control group 1 received only drinking water. Mice from groups 3 and 4 received intrarectally (via tubing) daily doses of 2 to 3×10⁹ CFU of F. prausnitzii HTF-F and A2-165, respectively in 100 μl PBS for ten days prior the DSS exposure and during the eight days of DSS treatment. Mice from group 5 received intrarectally daily doses of 50 μg the EPM in 100 μl PBS for ten days prior the DSS exposure and during the eight days of DSS treatment. Mice from the colitis control group received intrarectally 100 μl PBS. The following clinical symptoms were measured or assessed: firmness of faeces, rectal prolapses, rectal bleeding and colon length after the sacrifice. The colon descendens was taken for myeloperoxidase assay, isolation of mRNA, histological assessment and for intestinal fragment cultivation.

TABLE 1
DSS induced colitis experimental groups

| | Intrarectal | |
|---|---|---|
| Group | treatment | DSS |
| 1 | PBS | − |
| 2 | PBS | + |
| 3 | F. prausnitzii | + |
| | HTF-F | |
| 4 | F. prausnitzii A2- | + |
| | 165 | |
| 5 | the EPM | + |

Disease Activity Index

Disease activity index (DAT), measured according to Cooper et al. (Cooper, Murthy et al. 1993), is a combined score of weight loss, stool consistency and bleeding divided by 3. Acute clinical symptoms are diarrhoea and/or grossly bloody stools. The scores are explained in Table 2.

TABLE 2
Scoring of DAI (modified according to Cooper et al. 1993)

| | | Stool | Occult/gross |
|---|---|---|---|
| Score | Weight loss | consistency* | bleeding |
| 0 | None | Normal | Normal |
| 1 | 1-5% | Normal | Normal |
| 2 | 5-10% | Loose | Hemacult + |
| 3 | 1-20% | Loose | Blood in colon |
| | | | Starting bleeding from |
| | | | anus |
| 4 | >20% | Diarrhoea | Gross bleeding |

*Normal stools, well formed pellets; loose stools, pasty and semiformed stools which do not stick to the anus; diarrhoea, liquid stools that stick to the anus.

Histological Evaluation of Colon Damage

Colon tissue was fixed in Carnoy's fluid for 30 min, transferred into 96% ethanol and embedded in paraffin Five-mm paraffin-embedded sections were cut and stained with haematoxylin and eosin (H&E) and Alcian Blue and post-stained with NuclearFastRed (Vector, Burlingame, Calif.) for mucin production. Samples were examined using an Olympus BX 40 microscope equipped with an Olympus Camedia DP 70 digital camera, and the images were analysed using Olympus DP-Soft. The degree of damage to the surface epithelium, crypt distortion and mucin production in individual colon segments were evaluated according to Cooper et al. (Cooper, Murthy et al. 1993).

### Example 1

### Phenotypic Characteristics of F. prausnitzii Strain HTF-F and Purification of the Extracellular Polymeric Matrix

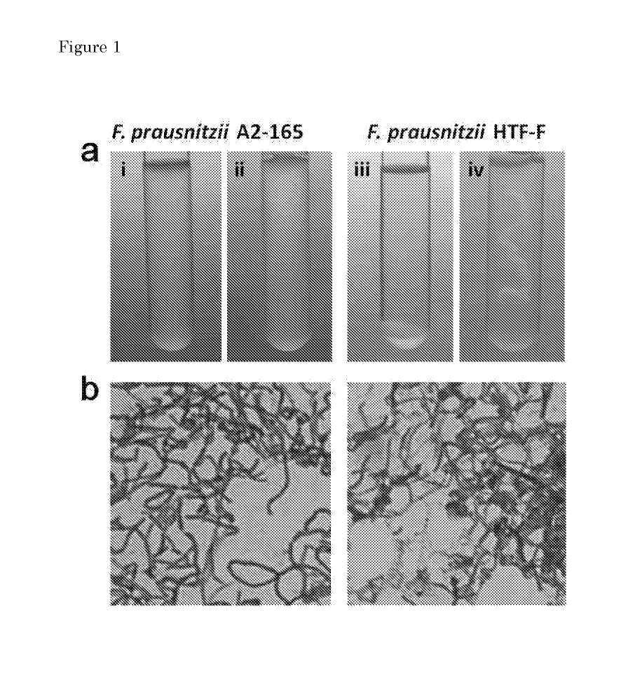

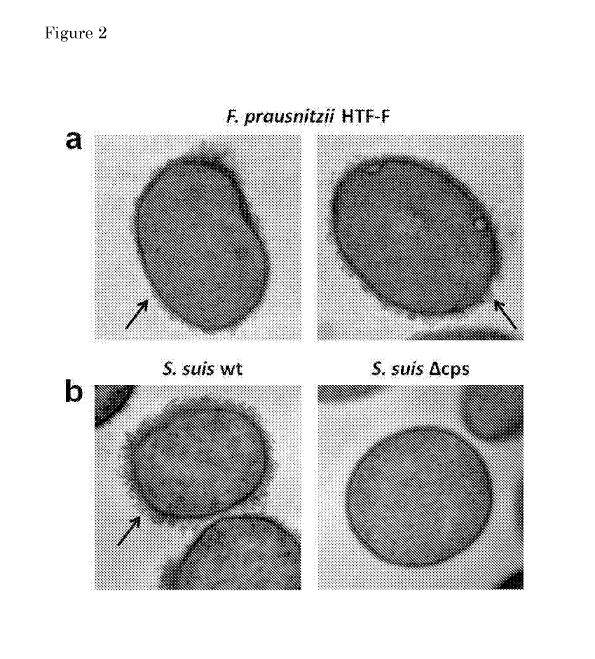

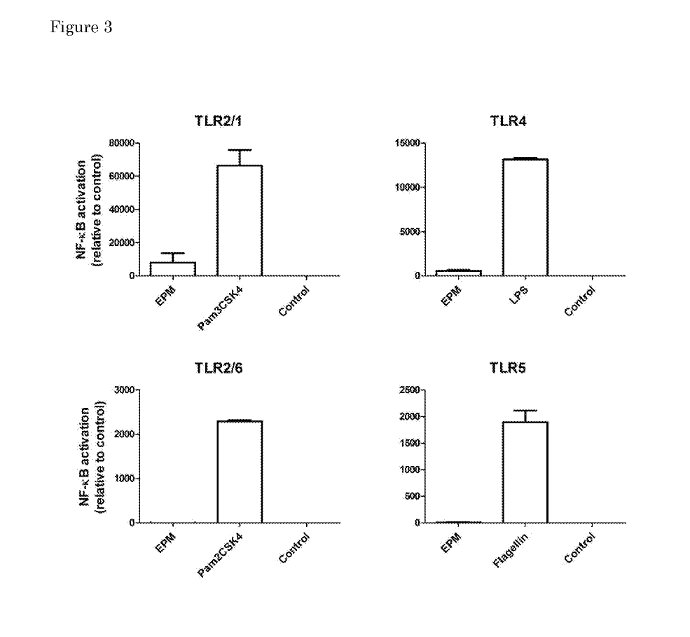

Although both F. prausnitzii strains HTF-F and A2-165 form colonies with mucoid appearance on solid agar, only strain HTF-F forms mucoid biofilm in liquid culture (FIG. 1a). This phenotype is commonly associated with the production of extracellular polysaccharides and intercellular aggregating proteins (Flemming and Wingender 2010). The EPM of strain HTF-F is revealed by Gram staining (FIG. 1b) and is observed in transmission electron micrographs as a diffuse and irregular surface layer (FIG. 2a, arrow) resembling the capsule polysaccharide (CPS) of Streptococcus suis (FIG. 2b, (Meijerink, Ferrando et al. 2012)). The cell bound the EPM produced by strain HTF-F was isolated, concentrated and filtered to remove possible bacterial contaminants. The EPM yield was 1.2 mg/ml from approximately 2.5×10¹¹-bacteria. Luciferase-based TLR signalling assays for human TLR2, TLR2/6, TLR4 and TLR5 indicated that microbe-associated molecular patterns (MAMPs) were not present in amounts that would influence activation of immune cells in vitro (FIG. 3). This was confirmed by the fact that the EPM did not induce activation or cytokine secretion after incubation with hDCs (FIG. 4b).

### Example 2

### The EPM of F. prausnitzii HTF-F Decreases Transcription and Production of Pro-Inflammatory IL-12p70 in L. plantarum-Activated hDCs

The immunomodulatory properties of F. prausnitzii A2-165, HTF-F and the EPM were tested in vitro using human monocyte-derived DCs (hDCs). DCs were chosen because they are one of the most important antigen presenting cells with the capacity to prime naive T cells at mucosal sites and to drive the immune response.

Incubation of hDCs with F. prausnitzii strains A2-165 and HTF-F induced large amounts of IL-10 and small amounts of IL-12p70 compared with L. plantarum (FIG. 4a) or other lactobacilli (not shown). The amount of IL-10 produced after incubation of hDCs with strain HTF-F was lower than with strain A2-165 (FIG. 4a). However, the difference is not significant. Moreover, the protective efficacy in vivo is more complex and may be dependent on additional factors, in particular strain survival and replication.

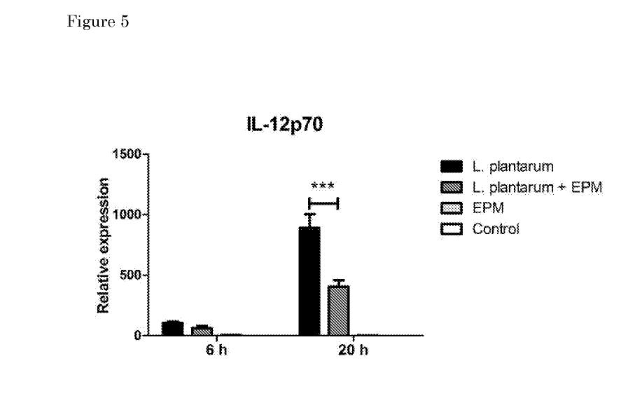

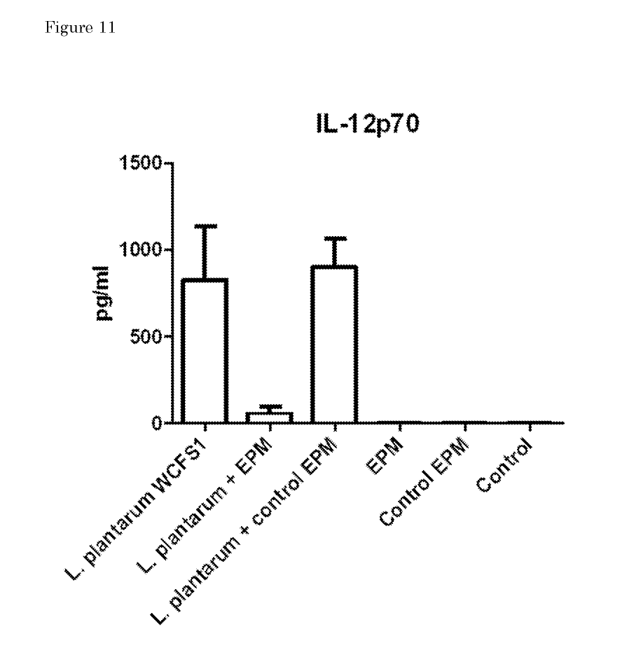

The EPM alone had no effect on the expression of activation or maturation markers and cytokine expression compared to untreated hDCs (FIG. 4b) confirming the lack of TLR signalling activity (FIG. 3). Therefore, the EPM was combined with L. plantarum in hDC cultures to investigate whether it would modulate cytokine production. In combination with L. plantarum, the EPM reduced the secretion of pro-inflammatory IL-12p70 but had no effect on the IL-10, IL-1β or TNF-α elicited by L. plantarum (FIG. 4a, IL-1β and TNF-α not shown). This was not due to an effect of the EPM on hDCs maturation and activation as evidenced by the measurement of the co-stimulatory molecules CD83 and CD86 (FIG. 4b). Thus, the EPM has an immunomodulatory effect on an inflammatory stimulus such as L. plantarum (shown in FIG. 4b, FIG. 5 and FIG. 11).

In order to investigate whether the reduced secretion of IL-12p70 was due to transcriptional regulation, quantitative RT-PCR was performed on IL-10, IL-12, IL-1beta and TNF-alpha mRNA extracted from hDCs at 6 and 20 h after incubation with L. plantarum, L. plantarum combined with the EPM, EPM alone or unstimulated hDCs. The addition of the EPM had no significant effect on transcription of IL-1beta, TNF-alpha or IL-10 in hDCs stimulated with L. plantarum (not shown) but significantly decreased the transcript levels of IL-12 at 20 h by about 2 fold (FIG. 5).

### Example 3

### F. prausnitzii and the EPM of Strain HTF-F Attenuate Clinical Symptoms in DSS-Colitis

The potential protective effects of F. prausnitzii strains A2-165, HTF-F and the EPM were assessed in mice using the DSS-induced colitis model. The bacteria or the EPM were administered to mice intrarectally ten days prior to DSS exposure and continuously administered daily over a period of eight days in which DSS was given in the drinking water to induce colitis. The severity of colitis was evaluated for individual mice in each group by measuring disease activity index (DAI), histological damage score of the colon, body weight and colon length. The DAI and the histological colon damage score after DSS treatment were assessed according to the scale (0-4) of Cooper et al. (Cooper, Murthy et al. 1993).

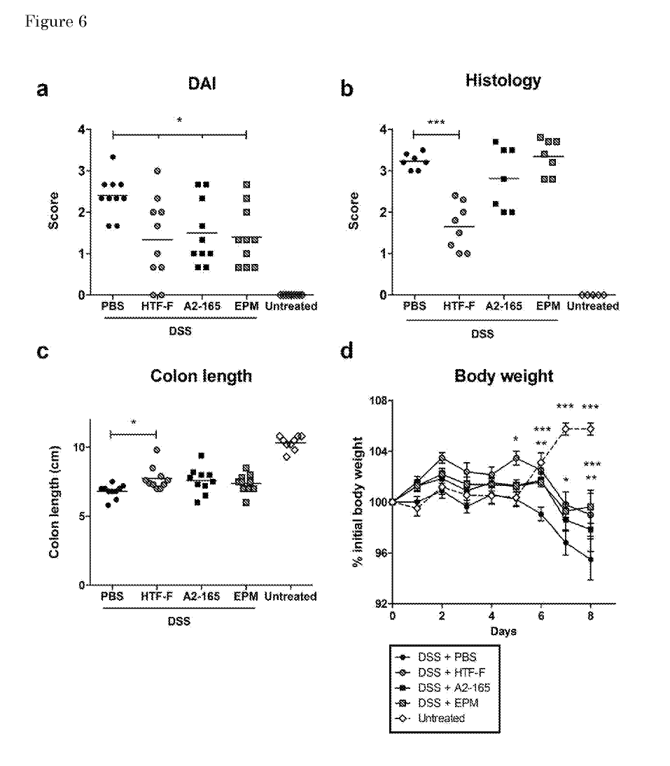

F. prausnitzii A2-165, HTF-F and EPM administration significantly decreased the DAI compared to colitis control mice which received PBS intrarectally and DSS in the drinking water, although the score was higher than in untreated mice (FIG. 6a).

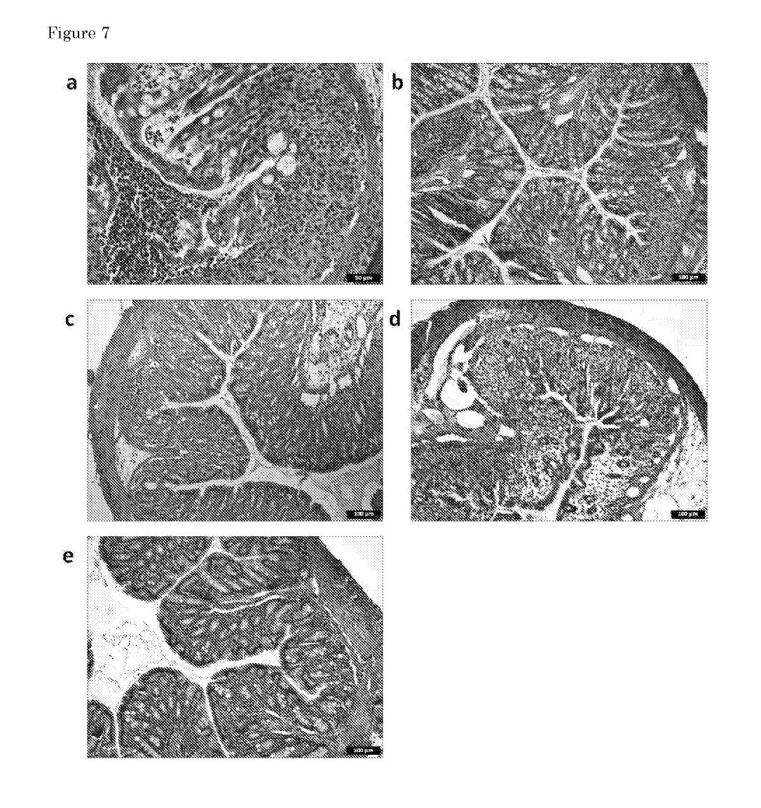

The histological colon damage score was grade 0 in untreated mice (FIGS. 6b and 7a). F. prausnitzii HTF-F administration significantly decreased colon damage score compared to the colitis control mice (grade 1.65 and 3.2 respectively, FIGS. 6b, 7c and 7b), while F. prausnitzii A2-165 and EPM administration did not significantly affect the colon damage score compared to colitis control mice (grade 2.8 and 3.3 respectively, FIGS. 6b, 7d and 7e).

The colon length was reduced in all DSS treated groups compared to untreated mice but the F. prausnitzii HTF-F treated group had significantly longer colons compared with colitis control mice (FIG. 6c) indicating reduced severity of colitis.

The body weight of mice was measured throughout the period of DSS treatment and compared to the weight before treatment. In untreated mice, the body weight increased by approximately 5% from day 5 to day 8 (FIG. 6d). However, in colitis control mice, the body weight decreased by approximately 5% from day 5 to day 8. In mice administered F. prausnitzii HTF-F, A2-165 or the EPM, the decrease in body weight was delayed by one day compared to the colitis control mice. In mice administered F. prausnitzii HTF-F, A2-165 and the EPM, the decrease in body weight from day 6 to day 8 was between 2 and 4% and for F. prausnitzii HTF-F and the EPM, it was lower than for the colitis control group (FIG. 6d).

Taken together, this example demonstrates that F. prausnitzii strain A2-165, the biofilm forming strain HTF-F as well as the EPM isolated from strain HTF-F can attenuate the clinical symptoms of DSS-induced colitis. Importantly, strain HTF-F was more effective than strain A2-165 in suppressing inflammation and having significant effects on the colon damage score and colon length compared to the other treatments (FIG. 6). The administration of purified EPM alone decreased the DAI indicating that it contributes to the protective effect of strain HTF-F and may be responsible for the stronger protection seen with strain HTF-F compared to A2-165.

### Example 4

### Effects of F. Prausnitzii and the EPM on Foxp3 Expression in Mesenteric Lymph Nodes and Spleen of DSS Treated Mice

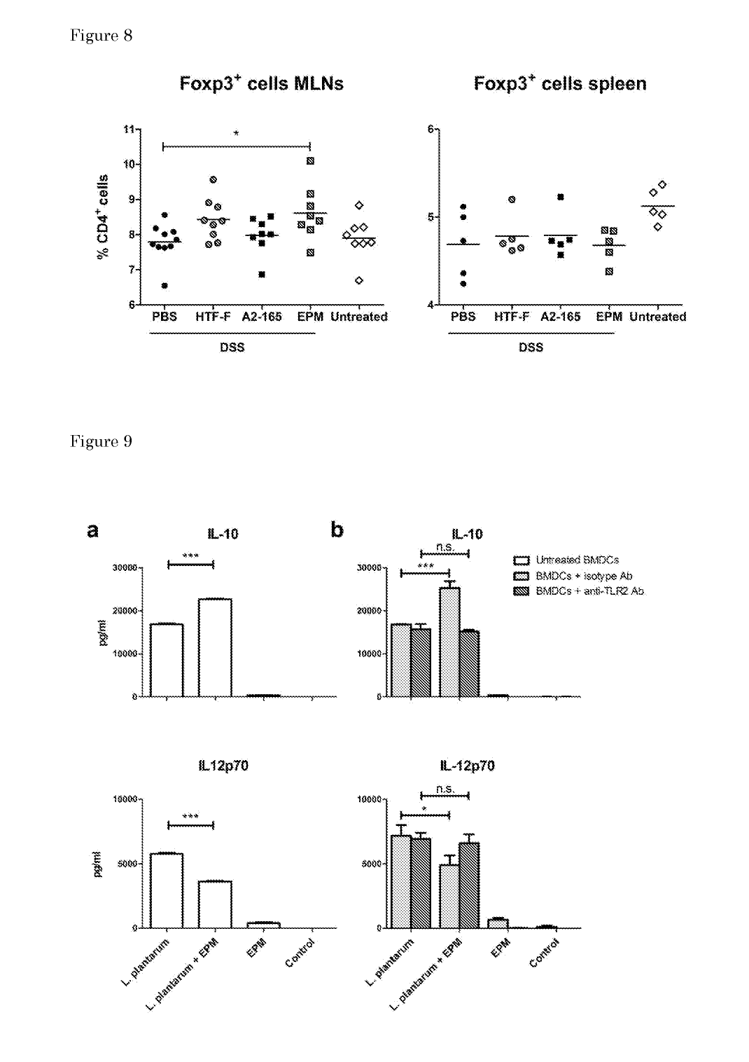

To investigate the potential role of Foxp3⁺ T regulatory cells (Tregs) in the attenuation of DSS-induced colitis, we measured the number of CD4⁺ T cells isolated from mesenteric lymph nodes (MLNs) and spleens that express intracellular Foxp3 by fluorescence-activated cell sorting (FACS). DSS treatment did not significantly affect the levels of Foxp3⁺ CD4⁺ T cells in the MLNs or spleen compared to untreated mice. Administration of F. prausnitzii also had no effect on Foxp3⁺ CD4⁺ T cells compared to untreated mice. However, administration of the EPM induced a small but significant increase in Foxp3⁺ CD4⁺ T cells in the MLNs but not in the spleen (FIG. 8).

### Example 5

### The Immunomodulatory Effects of the EPM are TLR2 Dependent

To investigate whether the anti-inflammatory effects of the EPM on IL-12p70 production by hDCs contribute to the protective effects observed in DSS-induced colitis, we performed an in vitro experiment using mouse bone marrow-derived DCs (BMDCs) stimulated with L. plantarum with and without EPM. As found using hDCs, the presence of EPM reduced the secretion of pro-inflammatory IL-12p70 by mouse BMDC stimulated with L. plantarum. Strikingly, the EPM also increased the production of IL-10 in BMDC stimulated with L. plantarum which was already induced at much higher levels than in hDCs stimulated with L. plantarum. These effects were not due to the induction of cytokine secretion by EPM alone (FIG. 9a). Additionally, we tested whether the immunomodulatory effects of the EPM were dependent on TLR2 signalling by including a TLR2 blocking antibody or an irrelevant antibody of the same isotype in the assays. The effects of the EPM on IL-12p70 and IL-10 production by L. plantarum stimulated BMDCs were inhibited in the presence of TLR2 blocking antibody but not in the presence of the isotype antibody. As EPM itself did not induce TLR2 signalling (FIG. 3) the mechanism leading to reduced transcription of IL-12p70 and increased production of IL-10 was dependent on the TLR2 signalling by L. plantarum (FIG. 9b).

Previously, F. prausnitzii A2-165 and its supernatant were shown to attenuate 2,4,6-trinitrobenzenesulfonic acid (TNBS) colitis in mice by daily intragastric administration prior to and during the induction of colitis (Sokol et al. 2008). In the study of Sokol et al., the colons of mice treated with either F. prausnitzii A2-165 or its supernatant had a reduced amount of IL-12p70 and an elevated amount of IL-10 compared with the colitis control group. This is compatible with the relatively high amount of IL-10 induced by in vitro culture of F. prausnitzii A2-165 with mouse BMDCs, hDCs (FIG. 4) and human PBMCs (Sokol, Pigneur et al. 2008). IL-10 is fundamental for the maintenance of homeostasis in the intestine (Geuking, Cahenzli et al. 2011; Veenbergen and Samsom 2012). It is secreted by DCs as well as Foxp3⁺ and Foxp3⁻ T cells in the lamina propria. Secretion of IL-10 by DCs is important for the maintenance of functional Foxp3⁺ Tregs during intestinal inflammation (Murai, Turovskaya et al. 2009). IL-10 also inhibits the production of pro-inflammatory cytokines such as IFN-gamma, TNF-alpha, IL-6 and IL-12. Moreover, IL-10 was shown to play a role in controlling pro-inflammatory responses to translocated microbes by abrogating IL-23 production (Manuzak, Dillon et al. 2012). Nevertheless, differences in IL-10 produced in vitro by DCs cultured with F. prausnitzii A2-165 and strain HTF-F cannot explain the better protection seen with HTF-F as it did not produce significantly different amounts of IL-10 than A2-165.

Butyrate produced in the colon by F. prausnitzii may also contribute to the anti-inflammatory effects observed in experimental colitis model, since oral administration of sodium butyrate has been recently shown to attenuate inflammation in experimental UC (Vieira, Leonel et al. 2012). Microbially-produced butyrate is considered important for colonic health, and in the prevention of colorectal cancer owing to its use as an energy source for epithelial cells and as a modulator of oxidative stress and inflammation (Hamer, Jonkers et al. 2008). Moreover, butyrate enemas have been reported to be effective in the therapy of UC (Hamer, Jonkers et al. 2010).

In vitro assays indicated that the anti-inflammatory mechanism of the EPM was not due to contamination with MAMPs or activation of DCs. However, when the EPM was added together with L. plantarum as an inflammatory stimulus to hDCs it decreased the production of IL-12p70 compared to L. plantarum alone and had no effect on IL-10, IL-1beta and TNF-alpha production (FIG. 4). A similar effect of the EPM was seen on IL-12p70 production by mouse BMDCs (FIG. 9) but, in contrast to hDCs, IL-10 was significantly increased. The effect of EPM on IL-12p70 production occurred at the transcriptional level (FIG. 5), which suggests the involvement of the EPM in cell signalling. This mechanism was dependent on TLR2 signalling although the EPM itself did not induce TLR2 signalling in reporter assays, or activate hDCs or mouse BMDCs as in the case of synthetic TLR2 agonists (FIG. 9). The mechanism may involve interaction of carbohydrate structures in the EPM with C-type lectin receptors, some of which are known to modulate cytokine production in response to TLR agonists. In the DSS colitis model, administration of EPM but not F. prausnitzii increased the number of Foxp3⁺ T cells in the MLNs.

### Example 6

### F. prausnitzii HTF-F Decreases the Secretion of IFN-γ and IL-17 from Colonic Cultures

Pre-weighted colonic fragments were cultured in RPMI medium enriched with 10% bovine serum albumin in 5% CO₂ and 95% air at 37° C., in 24-well flat-bottomed plates (Nunc, Roskilde, Denmark) for 48 h. Culture supernatants were harvested for analysis of their cytokine content by the MILLIPLEX MAP Mouse Cytokine/Chemokine Panel (Millipore, USA) according to manufacturer's instructions and analyzed with the Bio-Plex System (Bio-Rad Laboratories, USA).

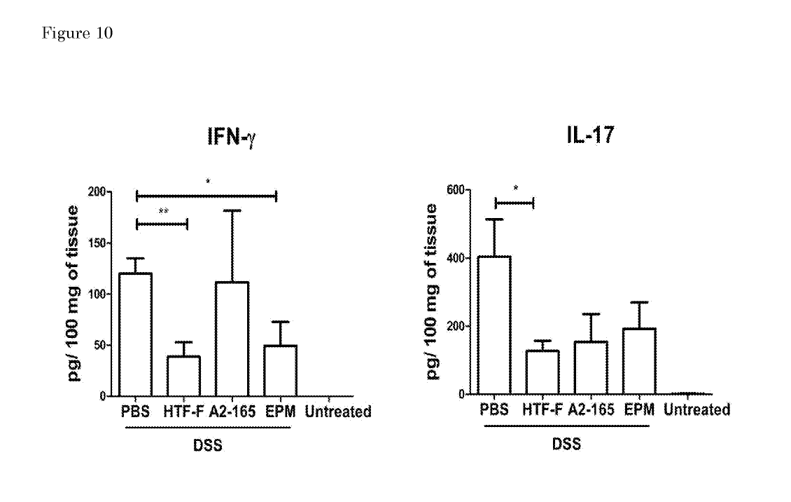

DSS treatment significantly increased cytokine secretion in colonic cultures compared to untreated controls. As shown in FIG. 10, intrarectal administration of F. prausnitzii HTF-F and EPM induced a decrease in the IFN-γ secretion and F. prausnitzii HTF-F induced a decrease in IL-17 secretion compared to PBS administration.

### REFERENCES

Barcenilla, A., S. E. Pryde, et al. (2000). “Phylogenetic Relationships of Butyrate-Producing Bacteria from the Human Gut.” Appl Environ Microbiol. vol 66(4): 1654-1661.
Cooper, H. S., S. N. Murthy, et al. (1993). “Clinicopathologic study of dextran sulfate sodium experimental murine colitis.” Lab Invest 69(2): 238-249.
Duncan, S. H. et al. (2002). “Growth requirements and fermentation products of Fusobacterium prausnitzii, and a proposal to reclassify it as Faecalibacterium prausnitzii gen. nov., comb. nov.” International Journal of Systematic and Evolutionary Microbiology 52(6): 2141-2146.
Fanning, S., L. J. Hall, et al. (2011). “Bifidobacterial surface-exopolysaccharide facilitates commensal-host interaction through immune modulation and pathogen protection.” Proc Natl Acad Sci USA 109(6): 2108-2113.
Flemming, H. C. and J. Wingender (2010). “The biofilm matrix.” Nat Rev Microbiol 8(9): 623-633.
Flint, H. J., K. P. Scott, et al. (2012). “Microbial degradation of complex carbohydrates in the gut.” Gut Microbes 3(4): 289-306.
Frank, D. N., A. L. St Amand, et al. (2007). “Molecular-phylogenetic characterization of microbial community imbalances in human inflammatory bowel diseases.” Proc Natl Acad Sci USA 104(34): 13780-13785.
Geuking, M. B., J. Cahenzli, et al. (2011). “Intestinal bacterial colonization induces mutualistic regulatory T cell responses.” Immunity 34(5): 794-806.
Hamer, H. M., D. Jonkers, et al. (2008). “Review article: the role of butyrate on colonic function.” Aliment Pharmacol Ther 27(2): 104-119.
Hamer, H. M., D. M. Jonkers, et al. (2010). “Effect of butyrate enemas on inflammation and antioxidant status in the colonic mucosa of patients with ulcerative colitis in remission.” Clin Nutr 29(6): 738-744.
Lopez-Siles, M., T. M. Khan, et al. (2012). “Cultured representatives of two major phylogroups of human colonic Faecalibacterium prausnitzii can utilize pectin, uronic acids, and host-derived substrates for growth.” Appl Environ Microbiol 78(2): 420-428.
Lutz, M. B., N. Kukutsch, et al. (1999). “An advanced culture method for generating large quantities of highly pure dendritic cells from mouse bone marrow.” J Immunol Methods 223(1): 77-92.
Macfarlane, G. T., K. L. Blackett, et al. (2009). “The gut microbiota in inflammatory bowel disease.” Curr Pharm Des 15(13): 1528-1536.
Manuzak, J., S. Dillon, et al. (2012). “Differential Interleukin-10 (IL-10) and IL-23 Production by Human Blood Monocytes and Dendritic Cells in Response to Commensal Enteric Bacteria.” Clin Vaccine Immunol 19(8): 1207-1217.
Mazmanian, S. K., J. L. Round, et al. (2008). “A microbial symbiosis factor prevents intestinal inflammatory disease.” Nature 453(7195): 620-625.
Meijerink, M., M. L. Ferrando, et al. (2012). “Immunomodulatory effects of Streptococcus suis capsule type on human dendritic cell responses, phagocytosis and intracellular survival.” PLoS One 7(4): e35849.
Murai, M., O. Turovskaya, et al. (2009). “Interleukin 10 acts on regulatory T cells to maintain expression of the transcription factor Foxp3 and suppressive function in mice with colitis.” Nat Immunol 10(11): 1178-1184.
Pfaffl, M. W. (2001). “A new mathematical model for relative quantification in real-time RT-PCR.” Nucleic Acids Res 29(9): e45.
Ricciardi, A., E. Parente, et al. (1998). “Use of desalting gel for the rapid separation of simple sugars from exopolysaccharides produced by lactic acid bacteria.” Biotechnology Techniques 12(9): 649-652.
Round, J. L. and S. K. Mazmanian (2010). “Inducible Foxp3+ regulatory T-cell development by a commensal bacterium of the intestinal microbiota.” Proc Natl Acad Sci USA 107(27): 12204-12209.
Schabussova, I., K. Hufnagl, et al. (2012). “Perinatal maternal administration of Lactobacillus paracasei NCC 2461 prevents allergic inflammation in a mouse model of birch pollen allergy.” PLoS One 7(7): e40271.
Seksik, P., L. Rigottier-Gois, et al. (2003). “Alterations of the dominant faecal bacterial groups in patients with Crohn's disease of the colon.” Gut 52(2): 237-242.
Sokol, H., B. Pigneur, et al. (2008). “Faecalibacterium prausnitzii is an anti-inflammatory commensal bacterium identified by gut microbiota analysis of Crohn disease patients.” Proc Natl Acad Sci USA 105(43): 16731-16736.
Sokol, H., P. Seksik, et al. (2009). “Low counts of Faecalibacterium prausnitzii in colitis microbiota.” Inflamm Bowel Dis 15(8): 1183-1189.
Veenbergen, S. and J. N. Samsom (2012). “Maintenance of small intestinal and colonic tolerance by IL-10-producing regulatory T cell subsets.” Curr Opin Immunol 24(3): 269-276.
Vieira, E. L., A. J. Leonel, et al. (2012). “Oral administration of sodium butyrate attenuates inflammation and mucosal lesion in experimental acute ulcerative colitis.” J Nutr Biochem 23(5): 430-436.

1. A pharmaceutical, neutraceutical or food composition comprising as active ingredient Faecalibacterium prausnitzii strain HTF-F (deposited with the DSMZ on Mar. 1, 2013 under the accession number DSM 26943), and an acceptable carrier, diluent or excipient.

2. Composition according to claim 1, comprising viable cells of Faecalibacterium prausnitzii strain HTF-F (DSM 26943).

3. Composition according to claim 1, comprising non-viable cells of Faecalibacterium prausnitzii strain HTF-F (DSM 26943).

4. Composition according to any one of claims 1-3, further comprising one or more prebiotics, preferably oligosaccharides, more preferably fructooligosaccharides, pectins and/or inulin.

5. Composition according to any one of claims 1-4, further comprising one or more probiotic bacteria, preferably selected from the group consisting of Bifidobacterium, Lactobacillus, Streptococcus and Saccharomyces and mixtures thereof.

6. Composition according to any one of claims 1-5, being a food composition, food supplement or neutraceutical.

7. Composition according to any one of claims 1-5, being a pharmaceutical composition, preferably formulated in an orally ingestible form, preferably in the form of a capsule, microcapsule, tablet, granule, powder, troche, pill, suspension or syrup.

8. F. prausnitzii strain HTF-F (DSM 26943) for use as a medicament.

9. F. prausnitzii strain HTF-F (DSM 26943) for use in a method for treating or preventing symptoms associated with an inflammatory disorder and/or microbiota disbalance of the gastrointestinal tract of a mammalian subject.

10. F. prausnitzii strain HTF-F for use according to claim 9, wherein said subject is a human.

11. F. prausnitzii strain HTF-F for use according to claim 9, wherein said subject is a domestic animal or an agricultural animal.

12. F. prausnitzii strain HTF-F for use according to any one of claims 8-11, wherein said bacterium is formulated to be administered to a subject in a therapeutically effective amount, preferably wherein the therapeutically effective amount of the bacterium is about 10exp6 to 10exp11 CFU/day.

13. F. prausnitzii strain HTF-F for use according to any one of claims 8-12 wherein said gastrointestinal disorder is selected from the group consisting of inflammatory bowel disease, Crohn's disease, irritable bowel syndrome, coeliac disease, infectious colitis, ulcerative colitis, and any combination thereof.

14. A method for producing an anti-inflammatory composition, comprising the steps of
a) harvesting living cells of Faecalibacterium prausnitzii strain HTF-F (DSM 26943) and resuspending the cells in a suitable buffer, preferably PBS;
b) vortexing the resuspended cells for at least 3 minutes allowing the cell-bound extracellular polymeric matrix (EPM) to dissolve in the buffer;
c) pelleting the cells by centrifugation and harvesting the supernatant
d) adding about 4 volumes of ice-cold ethanol to the supernatant to precipitate the EPM;
e) washing the precipitated EPM with ethanol;
f) optionally followed by lyophilization.

15. A synbiotic composition comprising living cells of Faecalibacterium prausnitzii strain HTF-F (DSM 26943) formulated with (i) riboflavin, riboflavin phosphate or a physiologically acceptable salt thereof in an amount of at least 0.05% based on the total dry weight of the composition, and (ii) cysteine, preferably present in an amount of at least 0.05% based on the total dry weight of the composition.

16. Synbiotic composition according to claim 15, further comprising inulin or inulin-type fructo-oligosaccharides, pectin preferably in an amount of 2-10% based on the total dry weight of the composition.

17. Use of Faecalibacterium prausnitzii strain HTF-F (DSM 26943) for the production of an anti-inflammatory composition.
